SEQUENCE LISTING (38 sequences)

SEQ ID NO: 1 - DNA, 240 bp, Homo sapiens
atggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggtaggtaaacttgactgcatgtttccaagtgggaattaa

SEQ ID NO: 2 - DNA, 156 bp, Homo sapiens
atggatgcagaattccgacatgactcaggatatgaagttcatcatcaaaaattggtacgtaaaataatttacctctttccactactgtttgtcttgccaaatgacctattaactctggttcatcctgtgctagaaatcaaattaaggaaaagataa

SEQ ID NO: 3 - protein, 79 aa, Homo sapiens
MDAEFRHDSGYEVHHQKLVFFAEDVGSNKGAIIGLMVGGVVIATVIVITLVMLKKKQYTSIHHGVVEVGKLDCMFPSGN

SEQ ID NO: 4 - protein, 51 aa, Homo sapiens
MDAEFRHDSGYEVHHQKLVRKIIYLFPLLFVLPNDLLTLVHPVLEIKLRKR

SEQ ID NO: 5 - protein, 44 aa, Homo sapiens
MVGGVVIATVIVITLVMLKKKQYTSIHHGVVEVGKLDCMFPSGN

SEQ ID NO: 6 - protein, 40 aa, Homo sapiens
MQNSDMTQDMKFIIKNWCSLQKMWVQTKVQSLDSWWAVLS

SEQ ID NO: 7 - protein, 20 aa, Homo sapiens
MQNSDMTQDMKFIIKNWYVK

SEQ ID NO: 8 - protein, 18 aa, Homo sapiens
MWVQTKVQSLDSWWAVLS

SEQ ID NO: 9 - DNA, 79 bp, Homo sapiens
ttgataattaaatgttatagcatggacactgacatttacattttttacttatgtttttggtttttaaatgactctgcat

SEQ ID NO: 10 - DNA, 37 bp, Homo sapiens
attattatttgaataatgaaattcatcagaacaatta

SEQ ID NO: 11 - DNA, 68 bp, Homo sapiens
gcaatttatagaaaaggaagagttcgtaggttataaattctgttagttgctaagaagcatttttaaaa

SEQ ID NO: 12 - DNA, 536 bp, Homo sapiens
aggttctgggtggacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggtaggtaaacttgactgcatgtttccaagtgggaattaagactatgagagaattaggcttagctttttgctaagaactagctaagtatctcttttaaaaaaccaatcagtgtgcttccatgatgcttgggttacagttgttctttcttgttttggttttcattcattgcaacttaccgtgaatattctgctcaaggtattgagagtgtgtgttgttatcttaacttacaatttgtgttgaagttatcaaataatacaaatgataatgcatgactttaaaaaagcat

SEQ ID NO: 13 - DNA, 262 bp, Homo sapiens
aggttctgggtggacaaatatcaagacggaggagatctctgaagtgaagatggatgcagaattccgacatgactcaggatatgaagttcatcatcaaaaattggtacgtaaaataatttacctctttccactactgtttgtcttgccaaatgacctattaactctggttcatcctgtgctagaaatcaaattaaggaaaagataaaaatacaatgcttgcctataggattaccatgaaaacatgaagaaataaataggctag

SEQ ID NO: 14 - DNA, 817 bp, Homo sapiens
ttgataattaaatgttatagcatggacactgacatttacattttttacttatgtttttggtttttaaatgactctgcattttgttttaagcttcaaattattatttgaataatgaaattcatcagaacaattagtgttaagaatcatatagcaatttatagaaaaggaagagttcgtaggttataaattctgttagttgctaagaagcatttttaaaattatgtactatagctctttattcagcagacgaaccaattacaatctgtgtaactagaacacttgatcaaaattatataatttttacaacgcttcactgcatagatacatgaacataatttattttgaattggaacaaagccccaaagtagcagttttgttctaccaggtaattaatgctcatttttaaaggcttttattattatttctgaagtaatgagtgcacatggaaaaagacacataataggctaaacaataagcccgtaagccaagccaacatattccaggaacaaatccttgccaacctctcaaccaggatttaacttctgcttttcccccattttcaaaaattatagcatgtatttaaatgcagcagaagccttactttcagggttcccttaccctttcatttctttttgttcaaaataggtagtaattgaaggtttaaatatagggtatcatttttctttaagagtcatttatcaattttcttctaacttcaggcctagaaagaagttttgggtaggctttgtcttacagtgttattatttatgagtaaaactaattggttgtcctgcatactttaattatgatgtaatac

SEQ ID NO: 15 - DNA, 59 bp, Homo sapiens
atgctcatttttaaaggcttttattattatttctgaagtaatgagtgcacatggaaaaa

SEQ ID NO: 16 - DNA, 38 bp, Homo sapiens
tattccaggaacaaatccttgccaacctctcaaccagg

SEQ ID NO: 17 - DNA, 26 bp, Homo sapiens
tagcatgtatttaaatgcagcagaag

SEQ ID NO: 18 - DNA, 56 bp, Homo sapiens
gaaggtttaaatatagggtatcatttttctttaagagtcatttatcaattttcttc

SEQ ID NO: 19 - DNA, 33 bp, Homo sapiens
ccaaataaagagcaagaataaagcaacatttca

SEQ ID NO: 20 - DNA, 22 bp, Homo sapiens
ttatgctttaaaaagcaataca

SEQ ID NO: 21 - DNA, 47 bp, Homo sapiens
tcctttctttcagaatgcctattcctgtgcattaaaagtgtccctcc

SEQ ID NO: 22 - DNA, 18 bp, Homo sapiens
tttaaagtaagcatcaaa

SEQ ID NO: 23 - DNA, 39 bp, Homo sapiens
ctttttatataacctcatccaaatgtcccctgcatttaa

SEQ ID NO: 24 - DNA, 41 bp, Homo sapiens
gaaaatgaaattcttctaattgcgtttataaattgtaatta

SEQ ID NO: 25 - DNA, 6 bp, Artificial Sequence
  Description of Artificial Sequence Synthetic oligonucleotide
aataaa

SEQ ID NO: 26 - DNA, 25 bp, Artificial Sequence
  Description of Artificial Sequence Synthetic primer
gtggacaaatatcaacaccgaggac

SEQ ID NO: 27 - DNA, 23 bp, Artificial Sequence
  Description of Artificial Sequence Synthetic primer
acatagtcttaattcccacttgg

SEQ ID NO: 28 - DNA, 24 bp, Artificial Sequence
  Description of Artificial Sequence Synthetic primer
gtcctgcatactttaattatgatg

SEQ ID NO: 29 - DNA, 26 bp, Artificial Sequence
  Description of Artificial Sequence Synthetic primer
agccatcatggaagcacactgattcg

SEQ ID NO: 30 - DNA, 25 bp, Artificial Sequence
  Description of Artificial Sequence Synthetic primer
gtggacaaatatcaagacggaggag

SEQ ID NO: 31 - DNA, 25 bp, Artificial Sequence
  Description of Artificial Sequence Synthetic primer
tccttaatttgatttctagcacagg

SEQ ID NO: 32 - DNA, 24 bp, Artificial Sequence
  Description of Artificial Sequence Synthetic primer
tcctgcatacctttaattatgatg

SEQ ID NO: 33 - DNA, 23 bp, Artificial Sequence
  Description of Artificial Sequence Synthetic primer
ttcatggtaatcctataggcaac

SEQ ID NO: 34 - DNA, 23 bp, Artificial Sequence
  Description of Artificial Sequence Synthetic primer
gtgttctttgcagaagatgtggg

SEQ ID NO: 35 - DNA, 23 bp, Artificial Sequence
  Description of Artificial Sequence Synthetic primer
acatagtcttaattcccacttgg

SEQ ID NO: 36 - protein, 18 aa, Homo sapiens
MVWQTKVQSLDSWWAVLS

SEQ ID NO: 37 - DNA, 433 bp, Homo sapiens
gtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggtaggtaaacttgactgcatgtttccaagtgggaattaagactatgagagaattaggcttagctttttgctaagaactagctaagtatctcttttaaaaaaccaatcagtgtgcttccatgatgcttgggttacagttgttctttcttgttttggttttcattcattgcaacttaccgtgaatattctgctcaaggtattgagagtgtgtgttgttatcttaacttacaatttgtgttgaagttatcaaataatacaaatgataatgcatgactttaaaaaagcat

SEQ ID NO: 38 - DNA, 807 bp, Homo sapiens
tatttttatgtttaatccaaataaagagcaagaataaagcaacatttcagattttggtttctggagacaatagttagaaagcatgagttatgattgacttaaaattcttgttgcctgtacttcactttgaaataacattatgctttaaaaagcaatacactgctaaaggttaatttgaattctgcagaattactatagcaaaaagtaggtaacaagatatctttttttctattgtttaactcctttctttcagaatgcctattcctgtgcattaaaagtgtccctccaaggaaattaggacatctgcagagttgaaaaacacctaagtctcagtcacttagagtcacacatcagggctcagagtgctatgactaggaaaatgctgacctcctttcattagtatgatcgttcctttccagcttttgatagatccaagcgctatcttcccaccactcaccaaatgttccacctgtcaaagggtttcaggtccctgcagacttcggttttgacctgtggggaaagtagacttcctcgaactggggaagccacatgttgtacatccttctataaactatgattatcattcttagtaggaaaatatgtgatttctttttttttttttttttttaaagtaagcatcaaatatttgaccaaccagttgggcagagaatatactgaaactttttatataacctcatccaaatgtcccctgcatttaagaaatgaaattcttctaattgcgtttataaattgtaaattatattgcatttagaaattaaaattctttttcttaatttgttttcaagg

(19) United States
(12) Patent Application Publication
(10) Pub. No.: US 2009/0192295 A1
(43) Pub. Date: Jul. 30, 2009

(54) AGENTS FOR PRE-SYMPTOMATIC DETECTION AND THERAPEUTIC TARGETING OF ALZHEIMER'S DISEASE AND DOWN SYNDROME IN HUMANS

(76) Inventors: Johanna BERGMANN, Hamburg (DE); Enrique Preddie, Montreal (CA)

Correspondence Address: FROMMER LAWRENCE & HAUG, 745 FIFTH AVENUE- 10TH FL., NEW YORK, NY, 10151, US

(21) Appl. No.: 12/273,639

(22) Filed: Nov. 19, 2008

Related U.S. Application Data
(62) Division of application No. 10/700,922, filed on Nov. 3, 2003, now abandoned.
Application No. 10/700,922 is a continuation of application No. 09/242,449, filed on Feb. 16, 1999, now abandoned.

(30) Foreign Application Priority Data
Aug. 22, 1996 (CA) 2,183,901

(51) Int. Cl. C07K 16/18

(52) U.S. Cl. 530/387.9

(57) ABSTRACT

Agents and methods for the diagnosis and therapy of Alzheimer's disease are disclosed. Such agents include three genes located within the region of human chromosome 21 occupied by the APP gene, which are exclusively expressed in Alzheimer's disease, respectively, the proteins encoded and expressed by these genes, the nucleic acid molecules influencing their expression, and endogenous antibodies produced in humans with Alzheimer's disease against the above proteins. Also disclosed are antibodies and nucleotides to detect the presence of the proteins and nucleic acids in humans.

This application is a divisional of U.S. patent application Ser. No. 10/700,922 filed Nov. 3, 2003, which is a continuation of U.S. patent application Ser. No. 09/242,449 filed Feb. 16, 1999, which is a National Phase Application of PCT/EP97/04599 filed Aug. 22, 1997 and claims priority to Canadian Patent Application 1 No. 2,183,901 filed Aug. 22, 1996.

### FIELD OF THE INVENTION

The invention relates to highly specific diagnostic markers, which are also highly specific targets for therapeutic reagents, which detect, prevent and treat Alzheimer's disease in humans More specifically, the invention relates to a unique protein encoded on human chromosome 21, within the locus found to be occupied by human APP gene, to analogues and derivatives of this molecule, and to nucleic acid molecules encoding such molecules or influencing their expression. The invention also relates to therapeutic methods for these molecules.

### BACKGROUND OF THE INVENTION

Alzheimer's disease (“AD”) is a progressive disease of the human central nervous system. It is manifested by dementia in the elderly, by disorientation, loss of memory, difficulty with language, calculation, or visual-spacial skills, and by psychiatric manifestations. It is associated with degenerating neurons in several regions of the brain.

The presence in postmortem brains of two proteins, hyperphosphorylated tau (PHF tau) and .beta.-amyloid (A.beta.), is the major pathological feature of all forms of human Alzheimer's disease (AD) and provides certain clues to some biochemical events in the disease. Normal tau is a cytoskeleton protein that functions as a microtubule “glue” in neurons, and .beta.-amyloid is a generally neurotoxic polypeptide fragment which is thought to be excised from the widely distributed APP transmembrane protein. PHF tau is present in the CSF of all humans with AD and A.beta. is present in CSF and blood platelets of people with AD. Price, D. L. et al. (Clin. Neuropharm. 14:S9-S14 (1991)); Pollwein, P. et al. (Nucl. Acids Res. 20:63-68 (1992)); Regland, B. et al. (Med. Hypoth. 38:11-19 (1992)) and Johnson, S. A. (In: Review of Biological Research in Aging, Vol. 4., Rothstein, M. (Ed.), Wiley-Liss, NY, 163-170 (1990)). Another significant clue to physiological processes which contribute to the etiology of AD is the presence of numerous inflammatory proteins in characteristic Alzheimer's lesions, (Rogers, J., Webster, S., Luc, L. F et al., Neurobiol. Aging 17:681-686 (1996).

Genes Predisposing to AD.

Four genes and a protein, viz. the APP gene on chromosome #21 (St. George Hyslop, P. et al, Science 235:885-890 1987; Goate, A. M. et al, Nature 349:704-706 1991) S182 gene on chromosome #14 (Sherrington, R. et al. Nature 375:754-760 1995), the STM2 gene on chromosome #1 (Levy-Lahad, E. et al. Science 269:973-977 1995) and the ApoE4 gene on chromosome #19 (Saunders, A. M. et al., Neurology 43:1467-1472 1993) has been definitely implicated in the etiology of the disease. The neuron specific cytoskeletal protein “tau” which is secreted into spinal fluid, is highly phosphorylated in AD and the latter form is found in quantities, related to the stage of the disease, in spinal fluid taken from AD patients. Although the function of tau, in the initiation of AD is not obvious, a parallel can be drawn with the behaviour of other microtubule proteins in epithelial cells which respond to transduction of membrane receptors by an unusual ligand. It has been shown that the interaction of enteropathogenic E. Coli (EPEC) within the membrane of cultured epithelial cells results in cytoskeleton rearrangement and hyperphosphorylation of three cytoskeletal proteins which migrate to a point below the site of membrane ligand interaction. The abnormally phosphorylated proteins form rigid channel-like structures that facilitates entry of bacterial virulent products into the cells (Rosenhine Llan, EMBO. J. 11: 3551-3560 (1992)) which are reminiscent of aggregates (NFTs) formed by tau in AD brains.

Mutations (several) in the APP gene segregate with an autosomal dominant, early onset form of AD in a few families (Goate, A. M. et al, Nature 349:704-706 1991; Selko, D. Scientific American 265:4047 1991; Hardy, J. et al. WIPO WO 92/13069 1992) mutations in S182, segregate with familial AD [FAD] (St. George-Hyslop, P, et al. Nature Genet. 2: 330-334 (1992); Sherrington, R. et al. Nature 375:754-760 (1995)); a mutation in STM2 segregates with a rare autosomal form of AD (Levy-Lahad, E. et al. Science 269:973-977 1995), and inheritance of the ApoE4 allele appears to be a susceptibility factor in all the above mentioned forms of AD (Corder, E. H. et al. Science 261:921-924 1993), while inheritance of the ApoE2 allele appears to confer a decreased risk (Corder H. L. et al. Nature Genet. 7:180-184 1994).

Nevertheless, the combined genetic factors account for only a small percentage of AD and, in spite of the identification of the latter factors, little is known or can be inferred about the biochemical cause of the disease symptoms. About 80% of all AD which occurs mainly in humans >60 years old are not obviously associated with genetic predisposition.

The Gene Products of Genes Predisposing to AD

I. The APP Gene on Chromosome 21

The APP gene is preferentially expressed in the neuronal cells of the central nervous system. The gene encodes a trans-membrane protein which contains a single monomeric trans membrane alpha helix (Kang J. et al. Nature 325:733-736 (1987); Tanzi, R. E. et al. Nature 321:528-530 1988)). The protein, which is expressed primarily in brain, can exist as several isoforms of various length caused by alternative splicing or by incorporating or shedding exons (Pollwein, P. et al. (Nucl. Acids Res. 20:63-68 (1992); Price, D. L. et al., Clin. Neuropharm. 14:S9-S14 (1991)), a feature it appears to have in common with the LDL receptor protein. However, all the isoforms have a common domain called the .beta. Amyloid domain (A.beta.), that is partly embedded within the membrane spanning region of the APP protein. The major product of post translational cleavage of APP protein is A.beta. (Podlisny, M. B. et al., Science 238:669-671 (1987); Currie, J. R. et al., J. Neurosci. Res. 30:687-689 (1991)). (Zain, S. B. et al., Proc. Natl. Acad. Sci. (U.S.A.) 85:929-933 (1988); Vitek, M. P. et al., Molec. Brain Res. 4:121-131 (1988); Johnson, S. A. (In: Review of Biological Research in Aging, Vol. 4., Rothstein, M. (Ed.), Wiley-Liss, NY, 163-170 (1990)). The secretion of the A.beta. protein thus reflects the cleavage of the domain from the precursor molecule (see, Roch, J. M. et al., J. Biol. Chem. 267:2214-2221 (1992)).

AS indicated above the pathological hallmark of AD (and Down syndrome, “DS”) is the presence of intracellular tangles, and extracellular deposits or “plaques” of amyloid protein A.beta. in the neuropil and in blood vessels (Mann, D. M. A. Neurobiol Aging 10:397-399 (1989); Selkoe, D. J. Neuron 6:487-498 (1991); Lampe, T. H. et al. Ann. Neurol. 36:368-378 (1994); Selkoe, D. J. Nature 375:734-735 (1995);). The principal component of the amyloid protein plaques in normal humans is A.beta., a 38-40 amino acid (.sup.about.4 kDa) hydrophobic protein (Price, D. L. et al., Clin. Neuropharm. 14:S9-S14 (1991)). A.beta. forms the core of fibrils, which are concentrated in amyloid deposits in the extracellular space of the brain parenchyma and in the vascular elements of the brain and the pia-arachnoid (Currie, J. R. et al., J. Neurosci. Res. 30:687-689 (1991)). However, in Alzheimer's disease a longer version of A.beta., A.beta.-42, is released from APP and it is this species of A.beta. which is believed to be the major component of amyloid deposits and is also believed to be the neurotropic factor in AD (Roher A. E. et al. Proc Nat Acad Sci USA 90:10836-10840 (1993); Games, D. et al Nature 373:523-527 (1995); Laferia, F. M. et al., Nature Genet 9:21-30 (1995)), and in DS (Iwatsubo, T. et al., Ann. Neurol. 37:294-299 1995; Neuron 13:45-53 1994). Nevertheless, the weight of experimental evidence is heavily against A.beta. as a neurodegenerative factor in vivo, (Irizarry, M. C., et. al., Neupopathol. Exp Neurol 56:956-973 (1997). Also, it has been demonstrated in mice that the presence of a functional APP gene is not necessary for the formation of AD type neuropathology (Zengh, H., et al. Cell 81:525-531 1995), and that synaptic loss can occur by overexpressing APP without plaque formation (Mucke, L. et al. Brain Res. 666:151-167 (1994)); nevertheless, the early build up of A.beta. in all forms of AD, (Querfurth, H. W. et al. Molec. Brain Res. 28:319-337 (1995); Levy-Lahad E., et al. Science 269:973-977 1995)), and the mutations in the APP gene which segregate with the disease, suggest a central role for APP in AD and DS (see also Hardy, John. Proc. Natl. Acad. Sci 94:2095-2097 (1997)).

The accumulation of A.beta.-42 in Alzheimer's disease is believed to result from the faulty processing of one or more of the APP isoforms (Currie, J. R. et al., J. Neurosci. Res. 30:687689 (1991)), (see also, Johnson, S. A. In: Review of Biological Research in Aging, Vol. 4., Rothstein, M. (Ed.), Wiley-Liss, NY, 163-170 (1990); Roch, J. M. et al., J. Biol. Chem. 267:2214-2221 (1992)). Such processing is thought to involve two or more specific proteases (Azuma, T. et al., J. Biol. Chem. 267:1609-1613 (1992); Nakanishi N. et al., Exp Neurol 121:125-(1993)). In spite of a concerted international effort by researchers to find these proteases, (“secretases”) they remain hypothetical to date.

II. Apolipoprotein E (ApoE) Located on Chromosome 19

ApoE proteins, like all soluble membrane proteins, aggregate in solution and ApoE4 is associated with A.beta. immunoreactivity in .beta. amyloid plaques. The latter might suggest that intra molecular protein aggregation could playa role in the pathogenesis of AD and DS. The finding that ApoE2 which has a cys residue at position 112, whereas ApoE4 does not, confers a decreased risk to AD to people having both alleles, suggest that a cys amino acid residue may confer the ApoE2 effect, and furthermore suggest that a cys residue may be involved in aggregation processes relevant to AD (Shi Du Yan et al., Nature 382:685-691 (1997); (Tabatin, M. et. al., Neurobiology of aging 17 (48) 5130 (1997))

III. S182 Located on Chromosome #14

Although the number of humans genetically predisposed to AD is relatively small, S182 is considered to be the major AD locus because the vast majority of Familial AD (FAD) patients have mutations in this region. The S182 gene encodes a transmembrane (TM) protein “AD3” which contains seven transmembrane helices. Five point mutations which occur in amino acid residues located in, or close to, different TM-helices, segregate with AD. Since the mutations are not found in normal humans they appear to be pathogenic for the AD phenotype in humans. The function of AD3 is unknown, but-possible functional homology with SPE-4, a transmembrane protein expressed in the worm Caenorhabditis elegans, might suggest that AD3 can function in the cytoplasmic partitioning of proteins (Sherrington, R. et al. Nature 375:754-760 (1995); Hardy, John. Proc. Natl. Acad. Sci 94:2095-2097 (1997)). Recent experimental evidence indicates that it might act as a receptor for APP.

IV. STM2 Located on Chromosome #1

Like S182, the STM2 gene encodes a TM protein containing seven transmembrane helices. The STM2 protein is highly homologous to S182, especially to sequences within the TM helices. A single point mutation in amino acid (aa) 141 located in the region of the second TM helix, which is 84% homologous to AD3 ™-II, was found to be the cause of AD in a large kindred family. As compared to FAD caused by mutations in S182, FAD caused by the STM2 141 mutation appears to be confined to one kindred, “The German Volga family”. The structural similarity between AD3 and STM2 protein indicates that they share a similar biochemical function which, as indicated above, is presently unknown (Levy-Lahad, E. et al. Science 269:973-977 1995). As in the case of S182it is likely to be a receptor for APP. Furthermore, the secondary structure of S182 and STM2 closely resembles that of the adrenergic and muscarine receptors. In the latter, several transmembrane domains are required for determining selectivity of antagonist and agonist binding (Wess, J. et al Mol. Pharmacol. 256:872-877 (1991)), and mutations within or close to these domains considerably diminish the binding of ligands to these receptors.

Without some type of effective treatment, AD will probably affect about one out of every 10 humans alive today. There is no effective treatment for AD at any stage of its clinical progression, and no reliable diagnostic method to detect the disease at early stages, even within the relatively small number of people genetically predisposed to the disease. However certain memory enhancing substances and especially anti-inflammatory substances can modulate and retard some symptoms of the disease if it is diagnosed early.

In view of the importance of diagnosing, predicting, and treating AD, affective means for achieving these goals are pressing. The present invention supplies such means.

### SUMMARY OF THE INVENTION

The object of the present invention was to provide highly specific and acurate markers for AD in humans. These molecules, in addition to being accurate predictive agents for the diseases, should also be targets for therapeutic substances which would prevent and stop the progress of AD in humans without undesirable side effects.

According to the invention the highly specific molecules which are markers for AD and also targets for therapeutic intervention have been provided. Therefore, this invention concerns agents and use of these agents as markers for early diagnosis and targets for therapeutic approaches for Alzheimer's disease and Down associated AD. Such agents include three novel neuropeptides implicated in Alzheimer's disease as well as naturally occurring variations of these molecules, and nucleic acid sequences encoding such molecules, or influencing their expression.

### BRIEF DESCRIPTION OF THE FIGURES

FIG. 1A. Nucleotide sequence of alzas (SEQ ID NO: 12).

FIG. 1B. (i) Amino acid sequence of ALZASp (SEQ ID NO: 3)

(ii) Amino acid sequence of ALZASp3 (SEQ ID NO: 7)

(iii) Amino acid sequence of ALZASp4 (SEQ ID NO: 16)

FIG. 1C. Nucleotide sequence of alzas1 cDNA (SEQ ID NO: 13)

FIG. 1D. Amino acid sequence of ALZASp1 (SEQ ID NO: 4)

FIG. 1E. Nucleotide sequence of the 5′ upstream regulatory region of alzas gene (SEQ ID NO: 14).

FIG. 1F. Nucleotide sequence of alzas2 cDNA (SEQ ID NO: 37).

FIG. 1G. (i) Amino acid sequence of ALZASp2 (SEQ ID NO: 36)

(ii) Amino acid sequence of ALZASp5 (SEQ ID NO: 5)

FIG. 1H. Nucleotide sequence of the 5′ regulatory region of alzas2 gene (SEQ ID NO: 38).

FIG. 2A. Organisation of alzas gene, comparison with organization of APP gene: products expected from constitutive cleavage of ALZASp and from mutations in regions in chromosome#21 encoding APP.

FIG. 2B. Organisation of alzas1 gene, comparison with organization of APP gene: products expected from constitutive cleavage of ALZASp1 and from mutations in regions in chromosome#21 encoding APP.

FIG. 2C. Organization of alzas2 gene, comparison with organization of APP gene: products expected from constitutive cleavage of ALZASp2 and from mutations in regions in chromosome #21 encoding APP gene.

FIGS. 3A, 3B & and 3C. A Expression of AD and DS related mRNA in human brain and lymphocyte: (A) amplification with primer pair “alz289-lane 1=DS brain, lane 2 & 3=normal brain, lane 4=hippocampus, lane 5=AD cortex, (only a small amount of the PCR product was applied to the gel) lane 6 & 7=DNA size markers, lane 7=normal lymphocyte, lane 7=AD lymphocyte; amplification with primer pair “alz188”-lane 1 & 4=DNA size markers, lane 2=AD brain, lane 3=normal brain.

FIGS. 4 A-B. ELISA methods

A. Description of the ELISA method used to detect ALZAS in human body fluids and tissue.

B. Description of the ELISA method used to detect endogenous immunoglobulins produced in blood and serum of patients with AD.

(C) Dot plots of serum from autopsy confirmed AD victims was probed with ALZab2 compared with serum from normal (no signs of AD) people.

(D) Detecting different levels of endogenous anti-ALZAS in serum from suspected early AD patients (e.g. patients with depression) and patients with clinical AD.

(E) Testing a scanning for pre symptomatic AD in clinically normal people >65 years old.

FIGS. 5A-D

(A). Isolation of ALZAS from serum obtained from a single autopsy confirmed AD patient. The protein was isolated using affinity purification methods on affinity purified anti ALZab2 antibody linked to CNBR-sepharose. The gels were stained with silver stains and western blotted. Human IG fragments were detected with mouse anti-human mABs.

(B) Silver stain of SDS gel: proteins obtained following affinity purification of ALZAS from serum from a late stage AD patient.

(C) Detection of ALZAS-IgG complexes present on gel of SB with anti-human IgG (Fc fragment).

(D) Western blot of cationic non SDS polyacrylamide gel following electrophoresis of serum from (a) a patient with sporadic AD, and (b) a patient with swedish mutation AD.

### DETAILS OF THE INVENTION

In detail, the invention provides a nucleic acid molecule, substantially free of natural contaminants, that encodes a protein selected from the group consisting of, alzas, alzas1 and alzas2. In particular, the invention provides the above-described nucleic acid molecule wherein the sequence is, SEQ ID NO:1 and SEQ ID NO:2.

SEQ ID:NO:1

5′ ATGGATGCAGAATTCCGACATGACTCAGGATATGAAGTTCATCATCAA
AAATTGGTGTTCTTTGCAGAAGATGTGGGTTCAAACAAAGGTGCAATCATT
GGACTCATGGTGGGCGGTGTTGTCATAGCGACAGTGATCGTCATCACCTTG
GTGATGCTGAAGAAGAAACAGTACACATCCATTCATCATGGTGTGGTGGAG
GTAGGTAAACTTGACTGCATGTTTCCAAGTGGGAATTAA 3′

SEQ ID:NO:2

5′ ATGGATGCAGAATTCCGACATGACTCAGGATATGAAGTTCATCATCAA
AAATTGGTACGTAAAATAATTTACCTCTTTCCACTACTGTTTGTCTTGCCA
AATGACCTATTAACTCTGGTTCATCCTGTGCTAGAAATCAAATTAAGGAAA
AGATAA 3′

The invention also provides a protein, substantially free of natural contaminants, selected from the group consisting of, ALZASp, ALZASp 1, ALZASp2. In particular, the invention provides the above-described protein having a sequence of, SEQ ID NO:3, SEQ ID NO:4, SEQ ID NO: 5. The invention also provides three associated hypothetical proteins having a sequence of SEQ ID NO:6, SEQ ID NO:7 and SEQ ID NO:8.

SEQ ID:NO:3

MDAEFRHDSGYEVHHQKLVFFAEDVGSNKGAIIGLMVGGVVIATVIVITLV
MLKKKQYTSIHHGVVEVGKLDCMFPSGN

SEQ ID:NO:4

MDAEFRHDSGYEVHHQKLVRKIIYLFPLLFVLPNDLLTLVHPVLEIKLRKR

SEQ ID:NO:5

MVGGVVIATVIVITLVMLKKKQYTSIHHGVVEVGKLDCMFPSGN

SEQ ID:NO:6

MQNSDMTQDMKFIIKNWCSLQKMWVQTKVQSLDSWWAVLS

SEQ ID:NO:7

MQNSDMTQDMKFIIKNWYVK

SEQ ID:NO:8

MWVQTKVQSLDSWWAVLS

The invention also provides a reagent capable of diagnosing the presence of a molecule selected from the group consisting of, alzas, a ALZASp-encoding nucleic acid molecule, alzas1, a ALZAS1-encoding molecule, alzas2, a ALZAS2-encoding molecule.

The invention particularly concerns the embodiments wherein the reagent is a protein (especially an antibody, or a fragment of an antibody, which is capable of binding to, ALZASp (amino acids 67-79), ALZASp1 (amino acid 18-51), ALZASp2 (amino acid 32-44), [hy1]ALZASp, [hy2]ALZASp and [hy]ALZASp2 and an antibody or fragment of an antibody that is capable of binding to ALZASp (amino acids 67-79) and ALZAS2 (amino acids 32-44).

The invention also provides a method of treating Alzheimer's disease, by providing an individual, in need of such treatment, an effective amount of an antibody or anti peptide substance against, ALZASp, ALZASp1, ALZASp2, [hy1]ALZASp, [hy2]ALZASp and [hy]ALZASp2 or of a reagent to block the activation of the promoters PALZ1-PALZ14 and other regulatory elements which program transcription of, alzas, alzas 1, and alzas2 mRNA, having the SEQ IDs, SEQ ID NO:9, SEQ ID NO: 10; SEQ ID NO: 11, SEQ ID NO: 15, SEQ ID NO:16, SEQ ID NO:17, SEQ ID NO:18, SEQ ID NO:19, SEQ ID NO:20, SEQ ID NO:21, SEQ ID NO:22,-SEQ ID NO:23 and SEQ ID NO:24.

SEQ ID:NO:9

5′ TTGATAATTAAATGTTATAGCATGGACACTGACATTTACATTTTTTAC
TTATGTTTTTGGTTTTTAAATGACTCTGCAT 3′

SEQ ID NO:10

5′ ATTATTATTTGAATAATGAAATTCATCAGAACAATTA 3′

SEQ ID:NO:11

5′ GCAATTTATAGAAAAGGAAGAGTTCGTAGGTTATAAATTCTGTTAGTT
GCTAAGAAGCATTTTTAAAA 3′

SEQ ID:NO:15

5′ ATGCTCATTTTTAAAGGCTTTTATTATTATTTCTGAAGTAATGAGTGC
ACATGGAAAAA 3′

SEQ ID:NO:16

5′ TATTCCAGGAACAAATCCTTGCCAACCTCTCAACCAGG 3′

SEQ ID:NO:17

5′ TAGCATGTATTTAAATGCAGCAGAAG 3′

SEQ ID:NO:18

5′ GAAGGTTTAAATATAGGGTATCATTTTTCTTTAAGAGTCATTTATCAA
TTTTCTTC 3′

SEQ ID:NO:19

5′ CCAAATAAAGAGCAAGAATAAAGCAACATTTC A 3′

SEQ ID:NO:20

5′ TTATGCTTTAAAAAGCAATACA 3′

SEQ ID:NO:21

5′ TCCTTTCTTTCAGAATGCCTATTCCTGTGCATTAAAAGTGTCCCTCC
3′

SEQ ID:NO:22

5′ TTTAAAGTAAGCATCAAA 3′

SEQ ID:NO:23

5′ CTTTTTATATAACCTCATCCAAATGTCCCCTGCATTTAA 3′

SEQ ID:NO:24

5′ GAAAATGAAATTCTTCTAATTGCGTTTATAAATTGTAATTA 3′

### DESCRIPTION OF THE PREFERRED EMBODIMENTS

The consensus of all experimental evidence suggests: (1) that APP protein (the .beta. amyloid precursor) plays a major role in the expression of AD disease phenotype regardless of origin of the initial biochemical trigger of the disease; the involvement of APP is manifested by the abnormal deposits of a variety of A.beta. molecules in crucial regions of the brain, (2) A.beta. is a 38-42, 4.6 kd polypeptide which is a fragment of the .beta. amyloid precursor protein (see Selkoe, D. J. Neuron 6:487-498 (1991), Nature 375:734-735 (1995); Masters, C. L. et al. Proc. Natl. Acad. Sci. USA. 82:4245-4249 1985)) (3) the presence of A.beta. in brain and cerebral arteries signals the beginning of AD, although A.beta. does not cause the neurodegenerative symptoms characteristic of AD; (4) the APP gene is not overexpressed in AD although it is overexpressed in DS because of the additional gene dosage, but the same might apply to other chromosome 21 associated genes that have no involvement in DS, and (5) in the meantime, there is neither a reliable non invasive method to detect AD in the early stages nor an effective method to treat the disease.

The success of the present invention came mainly from our realization that there must be other genes within the APP locus with association to the AD and DS, and that at least one of these genes must also have a .beta.-amyloid related component. Therefore, we used a procedure which we had successfully used to find alternative genes, which are putative causative factors of other “genetic diseases”, to search for such genes which might segregate with AD, within the locus encoding the entire APP gene on chromosome 21 and the regions that flank the gene.

We call these alternative genes “piggy-back genes”, and we refer to this technology as “disease gene discovery by positional searching” (DGDPS). Piggy-back genes are transcribed in any orientation within the chromosomal locus occupied by another gene.

DGDPS Procedure.

This procedure has an advantage over gene isolation by cloning from a genomic or cDNA library, because it overcomes three important drawbacks, (1) the possibility that some DNA sequences cannot be cloned by the conventional methods, (2) that some mRNA sequences are of such low abundance that they are not represented in the cDNA library, and (3) the products of some cloned sequences are highly toxic to bacterial or other hosts.

In general, first we identified a gene closely related to a gene already genetically linked to a certain disease, then isolated the mRNA transcribed from the gene from disease tissue or patient's blood, then synthesized cDNA from the isolated mRNA with reverse transcriptase then amplified the novel cDNA with specific primers which flanked the entire coding region of the cDNA, then we identified the cDNA from the size following electrophoresis on agarose gel, and finally isolated the unique cDNA from the agarose gel. This allowed us to select out the desired molecule, if it was expressed, without having to probe several million cDNA clones.

The fact that A.beta., which is present in all AD, is a part of the APP protein, and other indications from results of our work with some other neurodegenerative diseases, we concluded that the biochemical link between all forms of AD was the APP protein. We hypothesized that A.beta. was not directly generated from APP embedded in the membrane, rather it came either from “soluble” i.e., transported APP or from a different protein which was likely to be expressed in significant amounts in a disease specific manner. We called the protein “ALZAS” (Alzheimer's disease associated), and we predicted that ALZAS would be structurally closely related to APP and transcribed from within the same chromosomal location as APP. Furthermore we predicted that ALZAS would influence the phosphorylation of tau, would initiate the immunological reactions which lead to complement formation in neurons and would be present in body fluids (serum, saliva, urine) of people with AD. The aim of this invention was to find ALZAS in patients with AD. Following is a detailed description of positional searching as it applied to the present invention:

(1) We examined the sequenced regions within the APP locus on chromosome 21 and selected potential complete orf's, i.e. with acceptable translation initiation sequences (see Kozak, M. Nucleic Acid Res. 12:857-872 1984) and translation termination stop codons (TAA, TAG or TGA) in place,

(2) next we used the method of Bucher et al., J. Mol. Biol. 212; 563-578 (1990) to identify putative promoter regions associated with the orf within 100-1000 bp 5′ upstream of the translation initiation sequence and we identified potential poly-A addition signals (the consensus poly-A addition sequence is AATAAA (SEQ ID NO: 25)) within a region of .sup.about.1000 bp 3′ downstream from the stop translation codon of the potential orf,

(3) then, orf's fulfilling the above two characteristics were translated into putative protein sequences using the universal code,

(4) then we analyzed the putative protein with our proprietary computer assisted protein finger printing technology and obtained information about the potential biochemical characteristics of the deduced proteins,

(5) next the biochemical characteristics of the deduced proteins were correlated with known clinical symptoms of the AD and DS (Mann, D. M. A Neurobiol Ageing 10:397-399 1989), and with the biochemical characteristics of the disease reported (Selkoe, D. J. Neuron. 6:487498 1991),

(6) RNA encoding proteins with properties correlating with the disease characteristics were selected as potential disease related candidates,

(7) detection of the presence of transcribed mRNA sequences encoding the protein in a cell was done by Reverse transcriptase PCR (RT-PCR), (Mullis, K. B., Cold Spring Harbour Symp. Quant. Biol. 51:263-273 (1986); Saiki, R. K., et al., Bio/Technology 3:1008-1012 (1985); Mullis K. et al., U.S. Pat. No. 4,683,202; Erlich, H., U.S. Pat. No. 4,582,788; Saiki, R. et al., U.S. Pat. No. 4,683,194 and Mullis, K. B., et al., Met. Enzymol. 155:335-350 (1987), using the Stratagene RAP-PCR RT-PCR kit according to the manufacturer's instructions, with unlabelled primers to detect cDNAs encoding the deduced proteins in RNA isolated from frozen human brain and lymphocytes. RNA from frozen brains were extracted by grinding postmortem frozen brains in a tissue homogenizer in the presence of diethylpyrocarbonate (DEPC). Total RNA was isolated using the Stratagene micro RNA isolating Kit, and poly (A)+RNA was isolated using Stratagene Poly(A)+ Quick mRNA isolation kit following the manufacturer's instruction conditions recommended by the manufacturer. Forward and reverse PCR primers were prepared to regions flanking the entire protein coding region of the orf of the selected protein (see table 1 for sequence of the primers and for the size of the expected amplified product). The amplified cDNA was electrophoresed on agarose gels and the size was determined by comparison with DNA size markers which were electrophoresed alongside. To verify the sequence of the cDNA, the region of agarose containing the desired size cDNA was extracted into H2O, precipitated with ethanol and a portion was cycle sequenced using the primers in “12” and Perkin Elmer ampli-Taq on the Perkin Elmer 376 A DNA sequencer using a non radioactive method described by Liu, C. et al. Nucl. Acid. Res 21:333-334-(1993).

(8) To determine if the proteins were actually expressed, epitopes were identified within the amino acid sequence of the protein using the method of Hopp, T. P. and Woods, K. R. Proc. Natl Acad Sci. USA 78:3824-3828 (1981) and their sequences compared to sequences in databases; epitopes (see table #2) having no homologue within the public databases were selected and mono-specific polyclonal rabbit antibodies were prepared against these and purified by immunoaffinity chromatography on Pharmacia LKB, CNBr-activated sepharose 4B according to the recommendation of the manufacturer (see section #11 on Immunology, in Current Protocols in Molecular Biology (Vol 1) Ausubel, F. M. et al (ed) John Wiley & Sons NY. N.Y. 1991).

9. To detect if the deduced proteins were expressed in human material, proteins were isolated from AD, and normal tissue, by precipitating the homogenized tissue with >80% ammonium sulphate and subjected to SDSPAGE electrophoresis as described in Laemmeli, U.K. Nature 227:680-685 (1970)), (16) following which the separated proteins were Western blotted onto nylon membrane and treated with the affinity purified antibody or spotted onto positively charged membranes and treated as above. Interaction of the antibody with the protein bound to the membrane was visualized with a chemiluminescent kit purchased from BioRad Inc according to the manufacturer's instructions (also see Blake M. S. et al. Anal. biochem.

Three structurally related proteins were discovered by the DGDPS procedure these we call ALZASp (“ALZAS”), ALZASp1 (“ALZAS1”) and ALZASp2 (“ALZAS2”); The latter is a truncated version of ALZAS.

ALZAS has structural similarities to small pore-forming proteins. These proteins, found in a variety of organisms, penetrate cellular membranes and mediate membrane damage, usually at a very low effector/target cell ratio. ALZAS has the APP transmembrane signal; therefore, like APP, ALZAS can be translocated by the same route across membranes of the endoplasmic reticulum (ER) and the cytoplasm and can insert into the same sites as APP in plasma membranes.

The genes alzas, alzas1 and alzas2, and the protein encoded by these genes are described in the examples given below. Our discovery that these molecules: (i) were expressed in (100%) of brains, lymphocytes and blood obtained from humans with AD, (ii) elicited an antibody response in humans before clinical symptoms of the disease was detected, (iii) were not detected in normal individuals (normal=individuals below 30 years and individuals above 60 with no history of a neurodegenerative disease), and (iv) appeared in humans in accordance with the known incidence of AD in the population, (i.e., they were detected in 2 of 5 clinically normal people over the age of 65 who appeared suspect for AD). Strongly supported involvement of ALZAS molecules in the etiology of AD. Furthermore, it was a strong indication that these molecules could be used for presymptomatic diagnosis of AD, and furthermore, assuming that these molecules were involved in the pathology of AD they were targets for active and/or passive vaccines type therapeutics to prevent and stop the progress of clinical symptoms of AD.

The Molecules of the Invention

### Example 1

The ALZAS Transmembrane Protein.

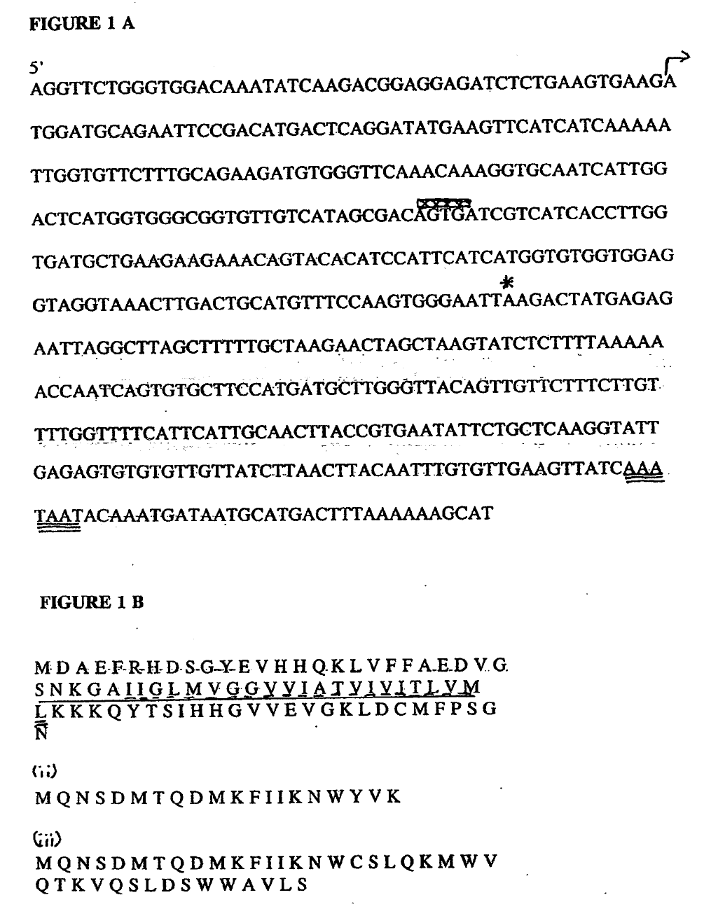

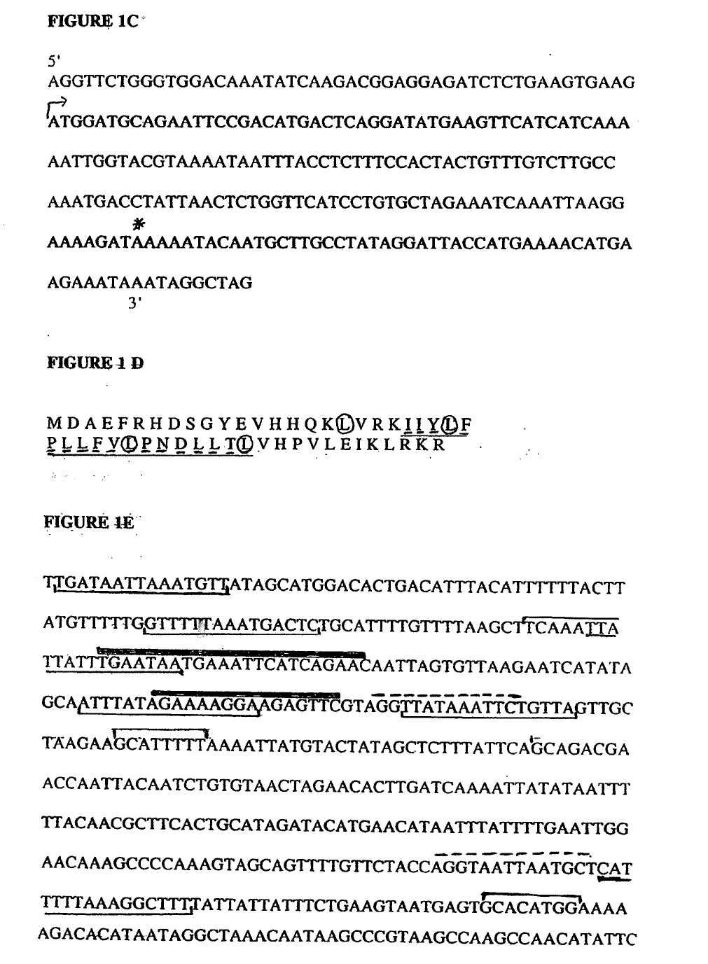

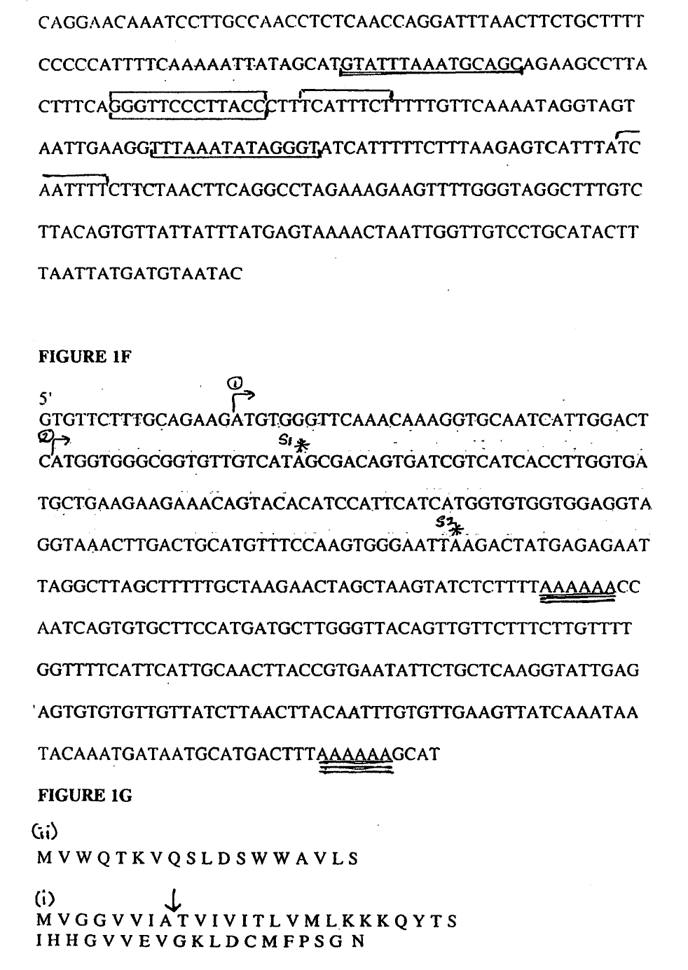

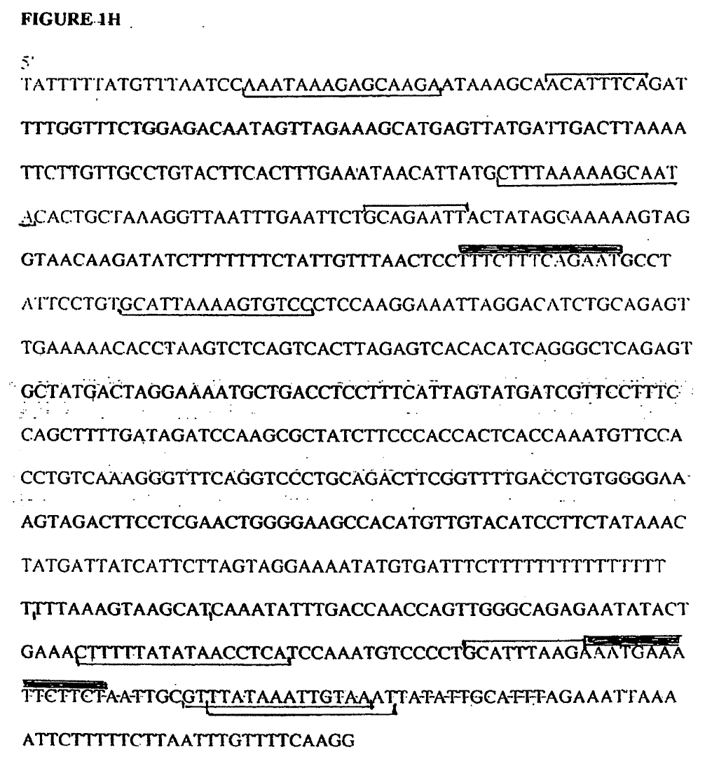

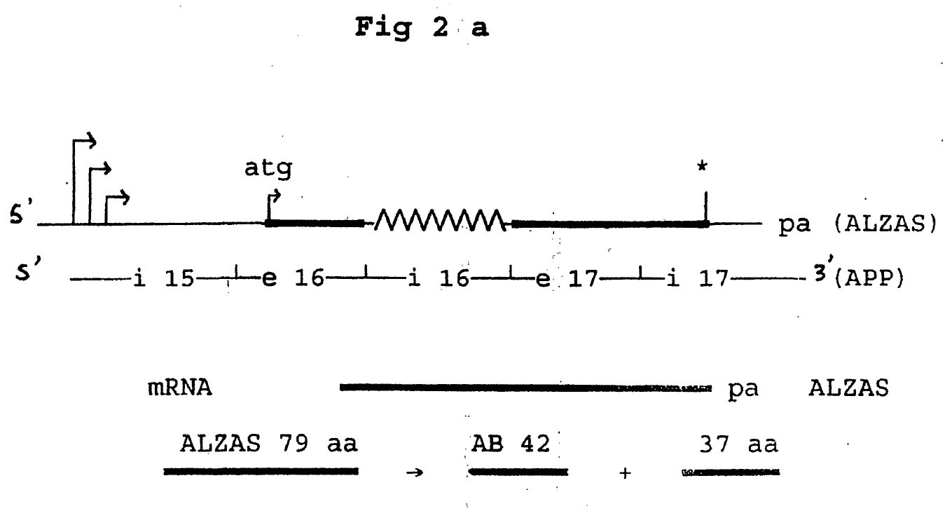

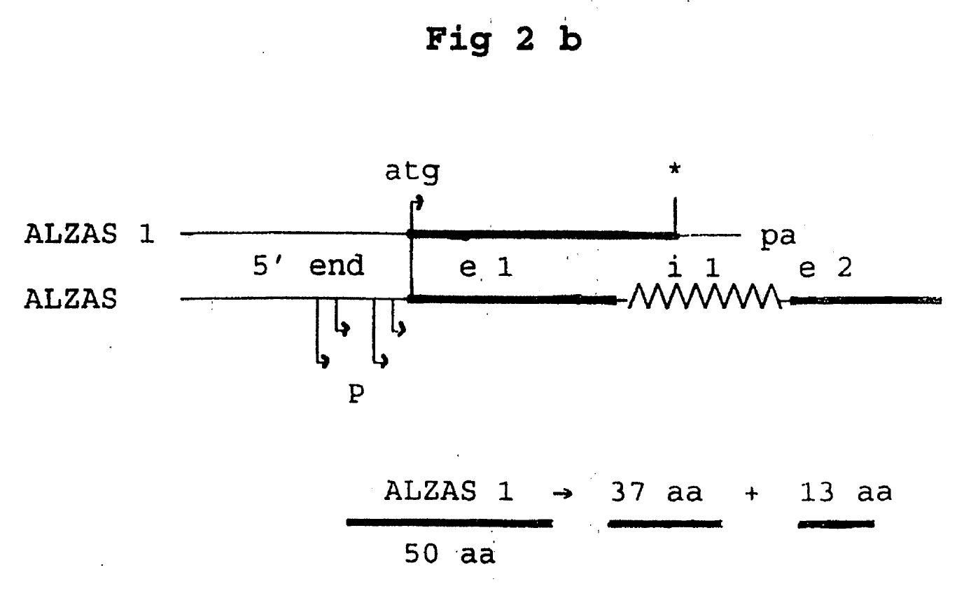

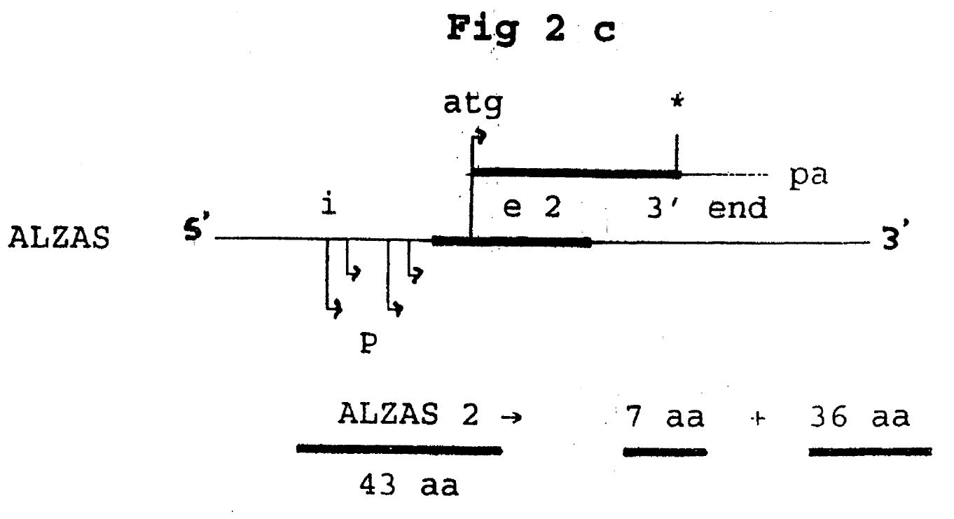

The positional relationship of the ALZAS family of proteins to APP encoding nucleotide sequences on chromosome 21 is shown in FIG. 2Ai. The gene alsas comprises two exons separated by a 5.6 kb intron. The nucleotide sequence of the transcription regulatory region of alzas, which lies within intron 15 of the APP gene, is shown in FIG. 1K. Transcription of alzas can be programmed by any of eight promoters, (“PALZ1”)+(“PALZ2”) SEQ ID:NO:9; (“PALZ3”) SEQ ID:NO:10, (“PALZ4”) SEQ ID:NO:11, (“PALZ5”) SEQ ID:NO:15, (“PALZ6”) SEQ ID:NO:16, (“PALZ7”) SEQ ID:NO:17 and (“PALZ8”) SEQ ID:NO:18, located in the regulatory region. PALZ3/4/5/6/7/8 are correlated with cap sites. Heat shock elements, which can modulate activity of all the alzas promoters, overlaps PAL3 (Heat shock proteins are reviewed by Gething, M. J. and Sambrock, J. Nature 355:33-45 (1992); two putative estrogen responsive elements (Savouret et al., Recent Progress in Hormone Res. 45:69-120 1989; Beato M. Cell 56:355-361 1989), lie ahead of and between PALZ 7/8. A potential poly-A addition site is present in the 3′ downstream untranslated region of the gene.

The alzas cDNA is shown in FIG. 1G SEQ ID:NO:1. It includes sequences identical to APP exon 16, exon 17 and part of intron 17, and encodes a 79 amino acid protein ALZASp, SEQ ID:NO:3, shown in FIG. 1Hi. The protein includes the complete A.beta. protein sequence, the APP transmembrane helix sequence (which has a constitutive hormone controlled secretory signal between aa 42//43) and a unique c-terminal sequence that is not related to APP amino acid sequences but has significant homology to a domain in a plant chloroplast membrane protein, and to the c-terminal sequence of ApoE4 proteins as determined with the method described by Feng, D. F. et al. J. Mol. Evol. 21:112-125 (1985).

How ALZAS Might Cause or Initiate AD in Humans.

The presence of a transmembrane helix (TM) identical to APP TM means that ALZASp (which is expected to have a far less complex secondary structure than APP mainly because of size) can successfully compete with APP for membrane anchor sites and may prevent APP from inserting into membranes. When this happens APP protein becomes vulnerable to degrading lysosomal protease activity. The latter could generate increased levels and various sizes of A.beta. protein fragments without any change in the actual level of APP mRNA transcription and APP protein expression in cells.

Computer assisted analysis of the secondary structure of ALZASp revealed four tandem amphipathic alpha helical regions. This helical organization, including the transmembrane helix, suggested that ALZASp might be related to membrane pore-forming proteins (Peitsch, M et al. Mol. Immunol 27:589-602 1990); (Hill, H. P. et al. Science 251:1481-1485 1991). By punching holes in a membrane, pore forming proteins cause slow dissolution of the membrane. This leads to increasing disruption, and eventual loss of function, of membrane proteins in the vicinity of the pore.

Stable interaction with another cellular protein can prevent oligomerization of a pore forming protein, and may prevent burial of the pore forming hydrophobic surface; the latter will have the effect of keeping the pore forming protein in the insoluble state; a condition which can effectively prevent the protein from reaching the membrane. In this regard, the evolutionary relationship between the c-terminal domains of ApoE proteins and the c terminal 12 amino acids of ALZAsp, indicated above, might be important. ApoE/2/3 interaction with ALZAS may become irreversible by forming an intra-molecular cys-cys bridge between, ApoE cys 112 and ALZASp, cys 73, that lies within the c-terminal domain. ApoE4 may not be able to interact irreversibly to ALZASp because it lacks the cys 112 residue, but may infact interact with and function as a chaperon for ALZAS to travers the cell's cytoplasm. This explanation is consistent with the increase probability of a double dose of ApoE4 allele on the chances of getting AD.

APP and presenilins appear to interact during maturation or transport of APP within intra cellular vesicles. By comparison with other multi-ligand, ligand-binding receptors (e.g. .beta.-adrenergic receptors, tachykinin receptors) AD3 and STM2 appear to be receptor sub-types. In these receptors the structural integrity of each TM helix is required for proper interaction between ligand and receptor (it has been demonstrated that several transmembrane domains are necessary to form the ligand binding pocket required for determining the selectivity of binding of ligands to adrenergic and muscarinic receptors (Wess J. et al. Mol. Pharmacol. 38:872-877 (1990)). This is one explanation for how mutations within or close to five TMs in AD3, and close to TM-II in STM2, may be pathogenic for AD. Thus a structural change in any of the interacting partners might decrease the critical level of interaction required between APP and presenilins and leave APP in an immature form, in cytoplasmic vesicles.

Structural and functional considerations indicate that ALZAS, in addition to the potential to mimic and replace APP in transmembrane transport and interactions, might also out compete APP for sites on PS1/PS2 in which mutations have altered the structural integrity of the a TM. Such structural change might increase the affinity of ALZAS for these sites while the affinity of binding APP is reduced. The results would be to increase the competitive advantage of ALZAS for blocking the membrane interaction between APP and PS1/PS2 ((Citron, M., et. al., Nature Medicine 3:67-72 (1997); Lamb Bruce. T. Nature Medicine 3:28-29 (−1997)). The end effect will be greater exclusion of APP from intracellular vesicles leaving this molecule to the mercy of all types of proteolytic action in intracellular vesicles and in the cytoplasm.

Furthermore affinity purification of ALZAS on columns of anti-ALZAS-sepharose columns of SDAT patients indicate that the ALZAS protein is bound to human immunoglobulin fragments in AD patients. This indicates that ALZAS is modulated by the immune systems in AD victims, and may be a target for complement derived destruction (see McGeer P. L. and McGeer, E. G. Ann NY Acad. Sci. 777:213-220 1996). The structural similarity between ALZAS and APP means that complement or other proteases directed at ALZAS may also inadvertently target A.beta. and the transmembrane sequence in APP protein. Joint proteolytic targeting of APP and ALZAS could explain all forms of A.beta., and can furthermore explain how A.beta. appears to be cut in a portion of the APP which lies within the TM signal. This also obviates a functional significance for specific digestion of APP by unique proteases. Therefore, it appears likely that the condition of a human immune response might play a significant role as to who gets AD and it puts into perspective the role of inflammation in the etiology of AD. Furthermore disruption of cytoskeletal structure which leads to abnormal phosphorylation of tau by activated tyrosine kinases may be a direct result of ALZAS interaction with the neuron plasma membrane (See Knulton J. et al., Infect. and Immuno., 57:1290-1298 (1989), Rosenshine et al., EMBO J. 11:3551-3560 1992)) just as production of multiple forms of A.beta. is due to the exclusion of APP from the membrane by competitive ALZAS binding and subsequent digestion by proteolytic enzymes.

Given the above indications, it appears that DS and AD may not be as closely related as we think. The factor which connects the two diseases i.e., the presence of A.beta., which may have little physiological significance in AD and DS is likely caused by two opposite mechanisms each resulting in production of A.beta. from free APP (i.e. associated with the membrane). In DS the accumulation of A.beta. may be due to the overproduction of APP, which over-saturates membrane sites and leads to exclusion of a significant amount of APP molecules in a cell; such excluded molecules, are digested in the cytoplasm. We suggest that in AD, APP is out-competed for membrane sites by ALZAS and remains in the cytoplasm where it is digested.

Amplification of Alzas in Human Frozen Brains and Lymphocytes

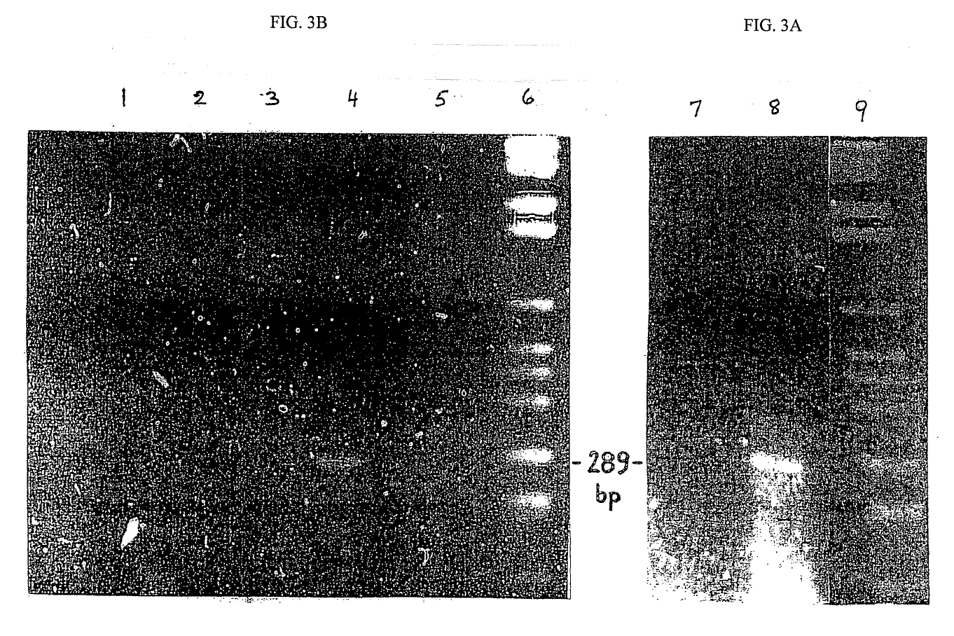

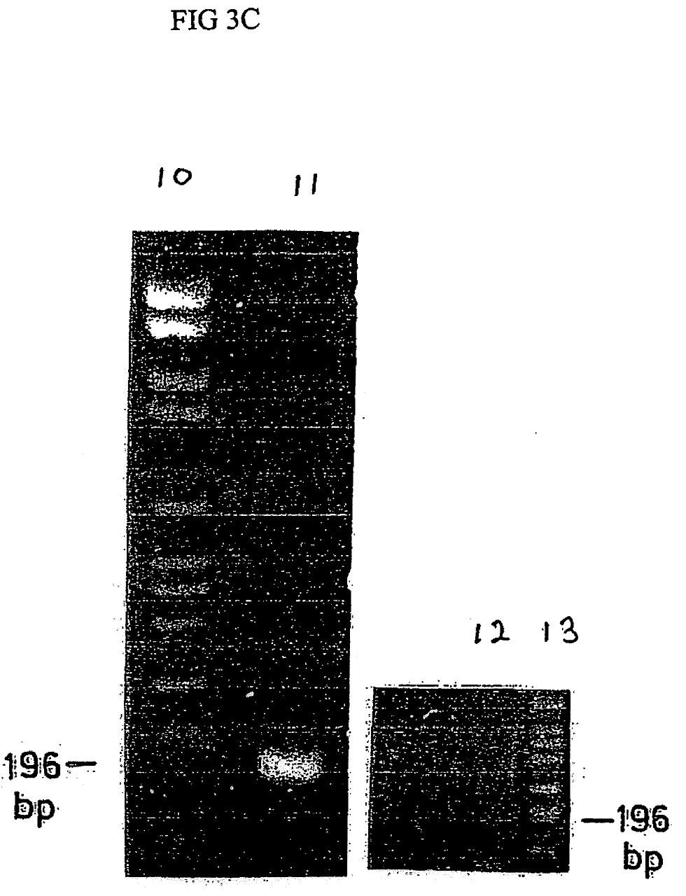

RNA was isolated from 13 frozen normal human brains and lymphocytes, frozen AD brains and lymphocytes. cDNA synthesis of mRNAs were done as described above and cDNA amplification was carried out with pp alz287 (see table 1a) (and with pp alz393 data not shown) which was expected to amplify a 287 bp fragment. The amplified fragment was detected by comparison with DNA size markers electrophoresed under the same conditions (FIG. 3A). Lane DS brain, lane 2 & 3 normal brain, lane 4, AD hippocampus, lane 5, AD cortex, lane 6 & 9 DNA size markers (Boheringer, marker #5), lane 7, normal lymphocyte, lane 8 AD lymphocyte. The amplified 287 base pair fragment was isolated from the agarose gel and subjected to DNA cycle sequencing using the nonradioactive method described previously for pp 287. The results (not shown) matched exactly the predicted nucleotide sequence which included, SEQ ID:NO:1.

TABLE 1A
PCR primers used to detect mRNA in lymphocytes and brain

| | | |
|---|---|---|
| alzas 287 | product size expected = | |
| | 287 bp | |
| Forward | GTGGACAAATATCAACACCGAGGAC | (SEQ ID NO:26) |
| Reverse | ACATAGTCTTAATTCCCACTTGG | (SEQ ID NO:27) |
| alzas 393 | product size expected = | |
| | 393 bp | |
| Forward | GTCCTGCATACTTTAATTATGATG | (SEQ ID NO:28) |
| Reverse | AGCCATCATGGAAGCACACTGATTCG | (SEQ ID NO:29) |
| alzas 188 | product size expected = | |
| | 188 bp | |
| Forward | GTGGACAAATATCAAGACGGAGGAG | (SEQ ID NO:30) |
| Reverse | TCCTTAATTTGATTTCTAGCACAGG | (SEQ ID NO:31) |
| alzas 267 | product size expected = | |
| | 267 bp | |
| Forward | TCCTGCATACCTTTAATTATGATG | (SEQ ID NO:32) |
| Reverse | TTCATGGTAATCCTATAGGCAAC | (SEQ ID NO:33) |
| alzas 148 | product size expected = | |
| | 148 bp | |
| Forward | GTGTTCTTTGCAGAAGATGTGGG | (SEQ ID NO:34) |
| Reverse | ACATAGTCTTAATTCCCACTTGG | (SEQ ID NO:35) |

Detection of ALZAS in Frozen Human Brains and Lymphocytes

Aliquots of proteins isolated, as described above, from frozen human brain, frozen AD brain, normal lymphocytes and AD lymphocytes, were spotted on a nylon membrane or subjected to western blotting as described, and probed with antibody ALZab2.

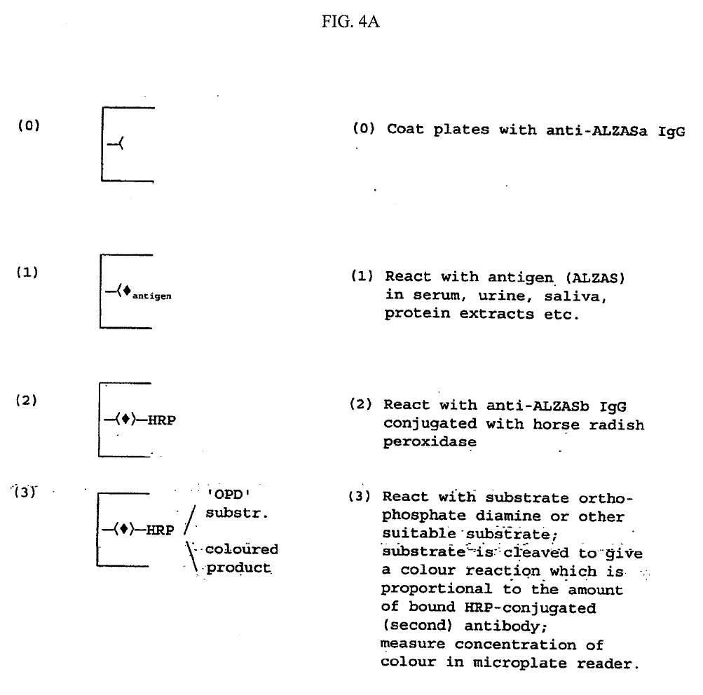

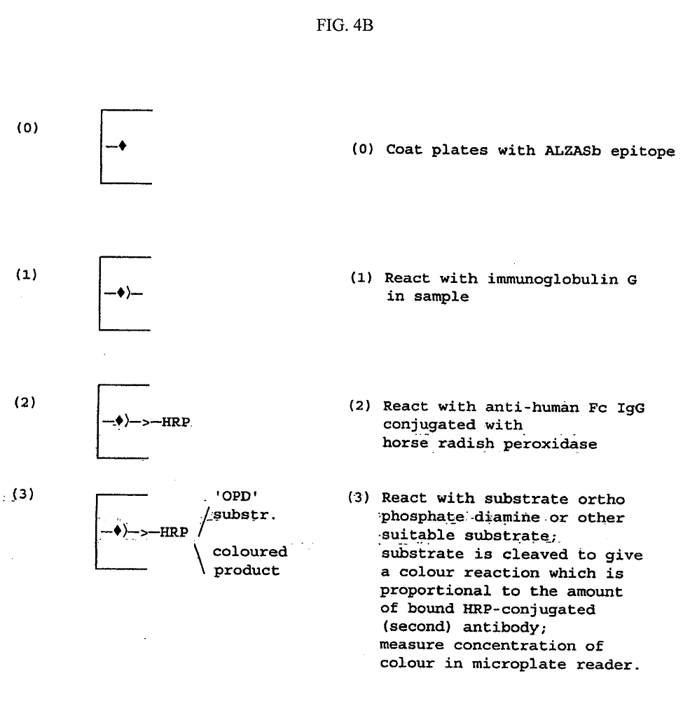

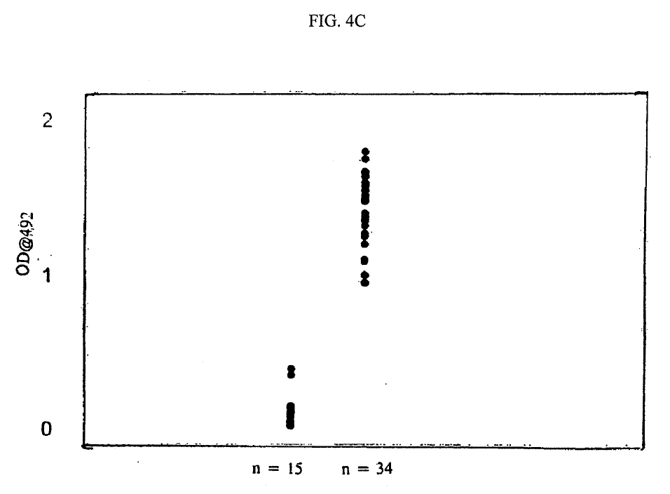

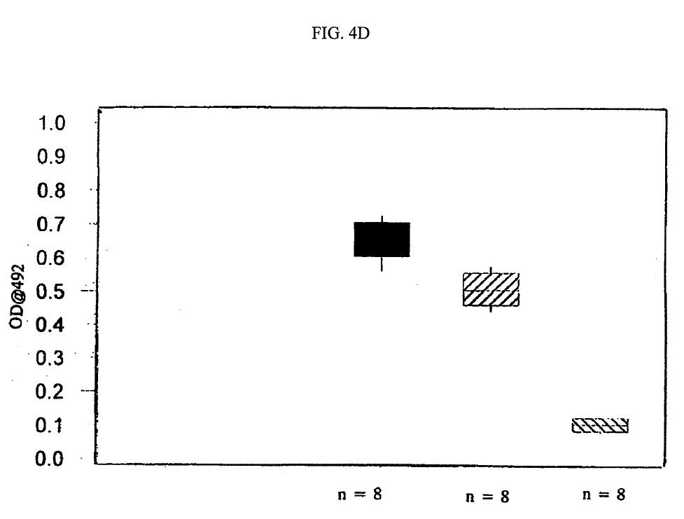

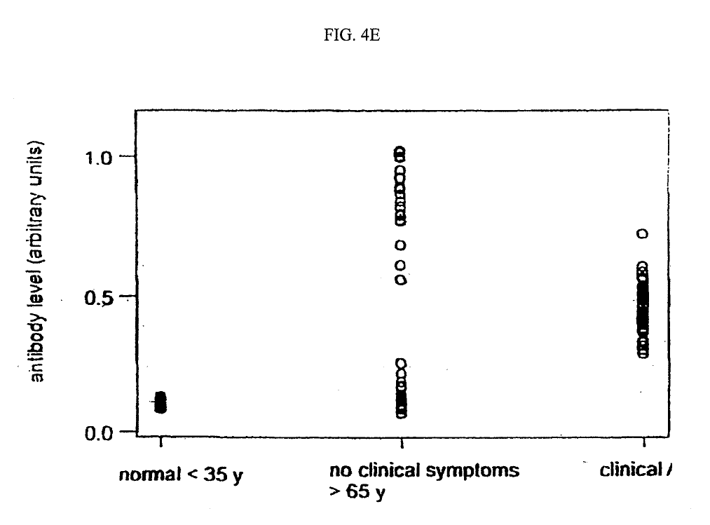

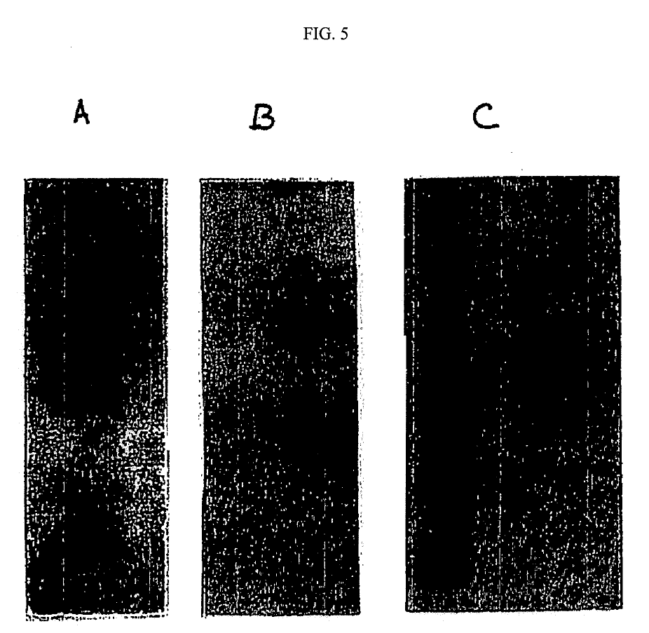

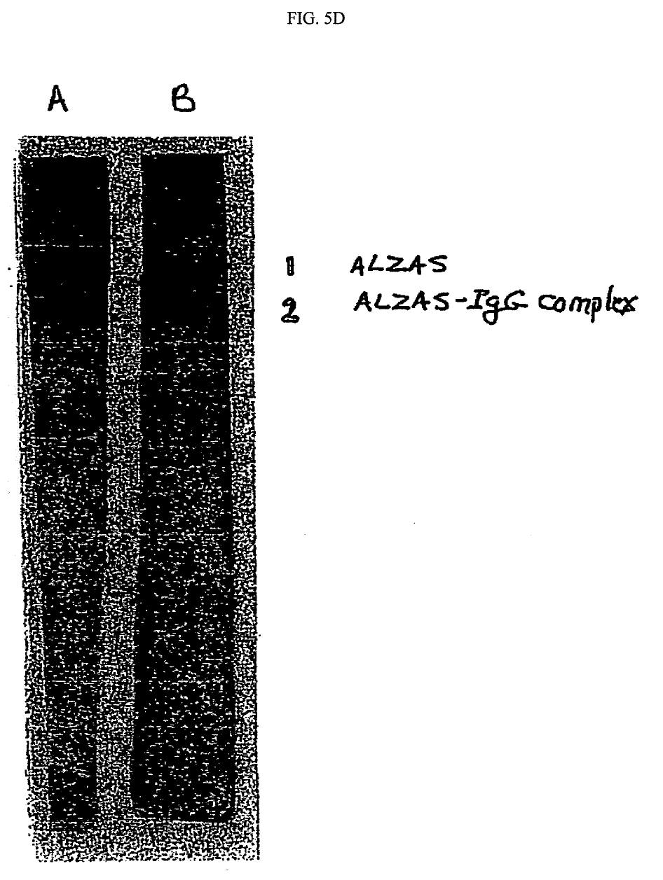

ALZab2 detects only the last 12 amino acids in the c-terminal of ALZAS. The results in FIGS. 4E & F show the presence of ALZAS in AD cortex and lymphocyte respectively. Western blots, FIG. 4D, lane 2 probed with ALZab2 showed a single, immunoreactive, band of 8.6 kd which was the expected size of ALZAS.

The presence of multiple promoter elements, heat shock elements, and putative estrogen receptor elements in the regulatory region of alzas is an indication that transcription of alzas mRNA might be triggered by a number of different factors including external environmental factors unrelated to mutations, e.g., stress inducing factors, metals, etc. However, certain factors might have a suppressive effect on the activation of the promoters (e.g estrogen, nicotinic acid related molecules, etc.,) and hence may delay, or even prevent, AD in some humans. Therefore, according to this invention these promoters are ideal targets for therapeutics which will block activation and prevent transcription of alzas in humans.

### Example 2

The ALZAS1 Membrane Associated Transcription Factor.

This invention also relates to a gene we call Alzheimer associated 1 (“alzas 1”). The location and organisation of alzas1 with respect to alzas is show FIG. 2B. The gene comprises an extended alzas exon 1. It shares the same 5′ regulatory sequences as alzas but it terminates within sequences homologous to APP intron 16. It has a consensus poly A site in the 3′ untranslated region.

The alzas1 cDNA, SEQ ID:NO:2, is shown in FIG. 1D. It encodes a 51 amino acid protein “ALZAS1” FIG. 1E, SEQ ID:NO:4. ALZAS1 is made up from the first 17 amino acids of A.beta. and 34 aa encoded by sequence homologous to APP intron 16. The protein contains a monomeric transmembrane helix and has a “leucine zipper”; it has a secretory signal which, if used, would releases the entire non membrane associated c-terminal domain. The c-terminal of ALZAS1 contain a five amino acid sequence which is identical to ApoE proteins heparin binding site and to the core sequence of ApoE LDL receptor binding sequence. This suggests that ALZAS1 might compete with ApoE proteins for binding to the LDL receptor and may be an important etiological factor in AD associated vascular diseases. It also provides additional indication that an evolutionary relationship might exist between ApoE proteins and ALZAS proteins.

Amplification of ALZAS1 in Human Frozen Brains and Lymphocytes.

PCR was done with pp alz188 using the same cDNA preparations from which alzas was amplified. The expected size of the amplified cDNA was 188 bp. significant amounts of the expected size cDNA was amplified from AD hippocampus FIG. 3C lane 2, whereas, considerable less cDNA was amplified from normal brain, lane 3 or from normal lymphocytes and normal brains, results not shown. Similar results were obtained by amplification with pp alz 267 (see table 1a).

Detection of ALZAS1 cDNA in Frozen Human Brains.

SDSPAGE of aliquots of protein isolated from frozen normal and AD brains (cortex) were transferred to nylon membrane and subjected to western blotting as described above and probed with ALZab3 (see table 1b). Immunopositive reactions were obtained with both normal and AD proteins but the reaction of the AD brain proteins, was considerably greater than that of the normal brain proteins, AD lymphocytes and normal lymphocytes. A 6.15 kd, protein, the predicted size of ALZAS1 was detected.

According to this invention alzas1 is overexpressed in AD and alzas is expressed only in AD. Since these genes share the same 5 upstream regulatory region, the signal that activates promoters transcribing alzas must also upregulate alzas 1 promoter/s. Therefore, ALZAS1 can potentially, compete with Apoe4 for interacting with LDL type receptors; as indicated earlier ApoE might play a chaperon role in mediating entry of APP (with which it might share a receptor binding site) through the plasma membrane. Therefore, APP entry into cells may be impeded by ALZAS1 compounding the effect of ALZAS proposed above. It follows then that ALZAS1 is a potential target for therapeutics which should prevent some of the symptoms of AD in humans.

### Example 3

The ALZAS2p Membrane Associated Protein.

The invention is also related to the discovery of a gene we call Alzheimer associated 2 (“alzas2”). The organization of the gene in relationship to alzas is shown in FIG. 2C. It is a single exon gene that is formed from an extended version of alzas exon 2. It is transcribed by six promoter elements (“PALZ9”) SEQ ID:NO:19, (“PALZ10”) SEQ ID:NO:20 (“PALZ11”) SEQ ID:NO:21, (“PALZ12”) SEQ ID:NO:22, (“PALZ13”) SEQ ID:NO:23 and (“PALZ14”) SEQ ID:NO:24, located in the 5′ upstream regulatory region of the gene. The sequence of the latter region is shown in FIG. 1G; it is homologous to sequences in APP intron 16. It harbours two potential heat shock elements, one upstream of PALZ11 and the other upstream of PALZ14, which may influence activity of the promoters.

The nucleotide sequence of alzas2 cDNA, SEQ ID:NO:37, is shown in FIG. 1F. There are two potential orfs in the cDNA; the most probable orf encodes a 44 amino acid protein ALZAS2, SEQ ID:NO:5, FIG. 1G. The aa sequence of ALZAS2 is identical to sequence 36-79 of ALZAS and can mimic all the activity of ALSAS related to a role in the pathology of AD, described above. Another hypothetical protein (“[hyp]ALZAS2p”) SEQ ID:NO: 36, which can be translated from alzas cDNA is shown in FIG. 1 Gii; protein fingerprinting indicates it may be a neuropeptide with a wide spectrum of physiological activity.

Amplification of Alzas2 cDNA in Frozen Brains and Lymphocytes.

pp alz141 which flanks the entire coding region of alzas2 was used to amplify the same preparations of cDNA used with pp alz287. No cDNA was amplified from DS lymphocytes, from AD lymphocyte, or from AD brain. cDNA was not amplified from five normal brain we tested. The failure to find transcripts for alzas2 in AD brains and lymphocytes is consistent with our expectation that alzas2 is transcribed only in those instances where FAD involves the DM to QL mutation in APP exon 16. ALZAS2, not ALZAS, is expressed in humans with this mutation (Swedish mutation) which is believed to be pathogenic for AD. The following example demonstrates that ALZAS2, not ALZAS, is involved in the pathology of this variety of FAD since ALZAS cannot be expressed in humans with this mutation, and ALZAS2 share the same antigenic site to which anti-ALZASa1b/a2b are directed.

Example: serum from a patient with sporadic AD, post mortem confirmed, and a patient from with the Swedish mutation, was subjected to cationic SDS-free polyacrylamide gel electrophoresis, western blotted and immuno-reacted with anti-ALZAS b. Positive reacting bands were visualized b subsequent treatment with the chemiluminescence anti-rabbit IgG system. As can be seen, in FIG. 5D the antibody reacted with protein bands in both the serum from sporadic AD and from the Swedish mutation AD. It can also be seen that the antibody interacted with a complex which we have found in other experiments, to be a complex of ALZAS and endogenous human anti-ALZAS IgG. The experiment demonstrated that ALZAS2 is a factor in the Swedish mutation AD and can be detected in humans with AD, and it indicated that, if indeed, ALSAS is an initiating factor in AD, only the transmembrane helix and the (intron encoded) unique c-terminal piece of ALZAS protein are required for initiating the disease.

In summary, ALZAS2 when expressed in humans can mimic some of the biochemical activities of ALSAS which might be involved in the pathology of AD.

Effect of Mutations in APP Exons 16 & 17 on the Expression of ALZAS Proteins.

There is a remote possibility that the region of chromosome 21 where alzas, alsas 1 and alzas2 are located may be a duplicate of a small region of chromosome 21. In this case, these genes may not be obligatory effected by mutations in the full length chromosome. However, it is more than likely that the genes are transcribed from within the APP gene, either in certain subsets of cells, or only when the promoters are sporadically activated by toxic intra/inter cellular factors produced in neuronal cells or toxic substances that enter such cells from the external environment. Other mutations in chromosome 21 which affect the configuration of DNA sequences in APP exons 16 and 17 and have been linked to early onset AD (Mullan, M. and Crawford, F. Trends neurosci. 16:398-403 1993), may occur also in the alzas genes. The Hardy VI and VG mutation, the VF mutation, and the Dutch EQ mutation that occurs in exon 17 (Selkoe, D. J. A Rev. Neurosci. 17:489-517 (1994); Hardy, J. Clin. Geriatr. Med. 10:239-247 1994)), cause a single amino acid change in ALZASp/p1/p2. However, the DM to QL mutation in exon 16 (Cai, X. D. et al. Science 259:514-516 1993) eliminates the normal orf for ALZASp and ALZAS1p which can lead to translation and expression of alternative proteins using other reading frames. Two hypothetical neuropeptides (“[hyp]ALZASp”) SEQ ID:NO:6, shown in FIG. 1Bii, and (“[hyp1]ALZASp”) SEQ ID:NO:7, shown in FIG. 1 Biii, can be translated from alzas cDNA. Amino acid 23-40 in [hyp1ALZASp] is identical to the sequence of [hyp]ALZAS2p. If expressed, the hypothetical proteins might play a role in the etiology of AD, and also lead to variations of the disease phenotype e.g., the mutation at codon #713 that causes schizophrenia, Jones, C. T. et al. Nature Gene. 1:306-309 (1992)), and the mutation at codon #702 that leads to hereditary cerebral haemorrhage, Dutch type (Levy, E. et al. Science:248 1124-1126 (1990)), may cause alterations in the biochemical property of at least one of the hypothetical proteins.

The Uses of the Molecules of the Present Invention.

A. Diagnostic Uses

ALZAS is expressed in all humans clinically positive for AD. ALZAS is also expressed in 2 out of 5 humans 65 years or older that appear clinically normal for AD (some of which have other neurodegenerative diseases) whereas ALZAS has not been found in humans that are distinctly normal, i.e. humans <50 years old without any visible symptoms or history of the AD. However, ALZAS has been found in saliva of menopausal women suffering from osteoporosis accompanied by depression; who had been treated with calcitonin. The detection of these molecules may be done by any of a variety of immunological methods (Yolken, R. H., Rev. Infect. Dis. 4:35 (1982); Collins, W. P., In: Alternative Immunoassays, John Wiley & Sons, NY (1985); Ngo, T. T. et al., In: Enzyme Mediated Immunoassay, Plenum Press, NY (1985); incorporated by reference herein.

In one embodiment, the affinity purified monospecific antibodies ALZab1, ALZab2, ALZab3 and ALZab4, based on the sequences in table 1b, (or selected amino acids within this sequence) can be used in any immune assay test system to detect, ALZASp, ALZASp1 and ALZASp2, e.g., in dot blot methods or in quantitative methods using sandwich ELISA and trap ELISA techniques as outlined in FIGS. 5A+B.

In another embodiment the presence of, alzas, alzas 1 and alzas2 mRNA in a cell or in the fluid as is described herein can be determined by any means capable of detecting mRNA encoding these proteins.

Such nucleic acid based assays may use either DNA or RNA to detect, alzas alzas1 and alzas2mRNA. In one embodiment, the assays may be performed on RNA that has been extracted from blood cells as described in the specifications herein. The assays may be done in situ on biopsied tissue using for example PCR (Mullis, K. B., Cold Spring Harbour Symp. Quant. Biol. 51:263-273 (1986); Saiki, R. K., et al., Bio/Technology 3:1008-1012 (1985); Mullis K. et al., U.S. Pat. No. 4,683,202; Erlich, H., U.S. Pat. No. 4,582,788; Saiki, R. et al., U.S. Pat. No. 4,683,194 and Mullis, K. B., et al., Met. Enzymol. 155:335-350 (1987), transcription-based amplification systems (Kwoh D et al., Proc. Natl. Acad. Sci. (U.S.A.) 86:1173 (1989); Gingeras T R et al., PCT appl. WO 88/10315; Davey, C. et al. European Patent Application Publication no. 329,822), etc.

B. Prognostic Uses

The present invention additionally provides a capacity to predict very early whether an individual has Alzheimer's disease. Thus, any of the above-described assays may be performed on an individual under treatment if a patient who has become asymptomatic restarts the process which leads to the restaging of the disease.

C. Therapeutic Uses

Significantly, the present invention provides a means for treating AD. Such treatment may be either “prophylactic” or “therapeutic.” A prophylactic treatment is one that is provided in advance of any clinical symptom of AD in order to prevent or attenuate any subsequent onset of the disease. A therapeutic treatment is one that is provided in response to the onset of a symptom of AD, and serves to attenuate an actual symptom of the disease.

In one embodiment, such treatment is provided by administering to a patient in need of such treatment an effective amount of an antibody, or an antibody fragment (F(ab′), F(ab′)2, single chain antibodies, etc.) that is capable of binding to, ALZASp (aa 67-79), ALZASp1 (aa 18-51), and ALZASp2 (aa32-44). The immunotherapy can be used in the form of an active vaccine, e.g. multiple antigenic epitopes based on the ALZAS sequence, or of a passive vaccine, e.g. humanized antibodies (see humanized antibodies in the following references which are incorporated by reference (Morrison, S. L., Science, 229:1202-1207 (1985); Oi, V. T. et al., BioTechniques 4:214 (1986); Jones, T. et al., Nature 321:552-525 (1986); Verhoeyan et al., Science 239:1534 (1988).

In another embodiment the desired therapy may be obtained by targeting the nucleic acid molecules, specifically the promoter sequences Palzas 1-14 of the present invention with anti DNA antibodies or “antisense” nucleic acid molecules. Antisense oligonucleotides are disclosed in European Patent Application Publication Nos. 263,740; 335,451; and 329,882, and in PCT Publication No. WO90/00624, all of which references are incorporated herein by reference. Used herein, an anti-sense nucleotide is a DNA or RNA whose sequence is complementary to the sequence of PALZ 1 to PALZ 14 described herein, such that it is capable of binding to, or hybridizing with, an endogenous promoter or heat shock sequence in a manner sufficient to impair its transcription, and significantly inactivate it in a cell; or whose sequence is complementary to alzas-nucleotide 201-240, or alzas1-nucleotide 55-156, or alzas2-nucleotide 96-135, and thereby impair (i.e. attenuate or prevent) its the translation into protein. These molecules may be transported into the cell using the Protein-Polycation Conjugates system (Beug, H. et al U.S. Pat. No. 5,354,844 Nov. 10, 1994) in an appropriate pharmaceutical compound, also see Oldham, R. K. (In: Principles of Biotherapy, Raven Press, NY, 1987), and Ledley, F. D., In: Biotechnology, A Comprehensive Treatise, volume 7B, Gene Technology, VCH Publishers, Inc. NY, pp 399-458 (1989)); all of which references are incorporated herein by reference.

The principles of the present invention can be used to provide a prophylactic gene therapy to individuals who, due to inherited genetic mutations, or somatic cell mutation, are predisposed to Alzheimer's disease.

Thus, in one embodiment of this invention, an antisense oligonucleotide that is designed to specifically block transcription or translation of a, alzas, alzas 1 or alzas2, mRNA transcript can be used to impair the expression of ALZASp, ALZAS1p or ALZAS2p in a cell, and thereby provide a treatment for AD.

III. Administration of the Molecules of the Present Invention

Additional pharmaceutical methods may be employed to control the duration of action of any of the aforementioned reagents used to treat AD. Such techniques are disclosed in Remington's Pharmaceutical Sciences (1980).

Puzzling Questions Answered by this Invention

Our data has led us to suggest certain physiological activities for ALZAS which links the many etiologies for the pathogenesis of AD e.g.: (i) Alzsas can compete with APP for insertion sites in intracellular (presenilins) and cell surface membranes (APP entry sites), by virtue of the identical transmembrane signal; this can disrupt neuronal cell signaling and cause some of the common physiological changes e.g., memory loss, characteristic of AD; (ii) ALZAS transported across the blood-brain barrier in leucocytes incorporates into the APP sites in selected neuronal cell plasma membranes; the 12 amino acid sequence (c-terminal immune response-eliciting toxic sequence) of ALZAS renders these cells targets for brain micro glial cells, and astrocytes producing an auto immune reaction which leads to an inflammatory type reaction which eventually kills the affected neurons; neuronal cells may recognize ALZAS attached to APP receptors as a pathogenic virulent factor of the ae ` `cmf` type and respond to contact with the protein by triggering phosphorylation of a specific cytoskeletal protein (TAU in this case) which assumes a new structural configuration, which is used to assist entry of the pathogen ALZAS, in this case, into the cell; ALZSAS might use apoe4 as a chaperon in order to traverse the cytoplasm. This is more efficiently with apoe4 which does not have a cys residue which can combine ireversely with the cys residue in ALZAS. ApoE2 and E3 have free cys residues therefore they might bind irreversible to ALZAS and prevent interaction of free ALZAS with APP membrane binding sites. Furthermore it provides a clearer insight into the generation of multiple size species of A.beta. in neurons.

Having now generally described the invention, through references and examples that makes it more readily understood by any one sufficiently skilled in the art, it must be pointed out that these are not intended to be limiting of the present invention, unless specified.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features herein before set forth and as follows in the scope of the claims.

### FIGURE LEGENDS

FIG. 1
ATG=open reading frame
*=stop translation signal
P=poly A addition signal
pck=protein kinase phosphorylation site
ck=casein kinase phosphorylation site
downwards pointing arrow=secretory/cleavage site
single overline=cap site
double overline=GC promoter element
double overline and underline=CCAAT promoter element
single underline=TATA promoter element
LxxxxxxL=leucine zipper
ooooooo=heat shock element (HSE)
[ ]=transmembrane signal
xxxxx=iron binding site
[C/H—C/H] . . . [C/H—C/H]=potential zinc finger=estrogen. responsive element (ERE)

FIG. 2
=promoters.
atg=orf
*=stop translation signal
p=poly A addition signal
-+-=size of fragments expected if potential cleavage site is used
   =sequence in alzas identical to sequence in ALZASp.
i=intron
e=exon

FIG. 3

### PCR Amplification:

A. mRNA isolated from postmortem AD and non AD tissues using alz 287 primer pair lane 1=DS brain, lanes 2-3=normal brain; lane 4=AD cortex; lane 5=AD cerebellum; lane 6=DNA markers; 7=normal lymphocyte; 9=AD lymphocyte; 10=DNA markers
B. using primer pair alz 148, lane 10&13=DNA markers; lane 11=AD cortex; lane 12=normal brain

FIG. 4

A=description of antigen trap ELISA test
B=description of antibody trap ELISA test
C=Dot plots of ELISA values for endogenous anti-ALSAS IgG measured in serum from 34 autopsy confirmed AD victims and 15 normal subjects. The values were not corrected for the normal background of the test.
(D) Box plots of ELISA values for endogenous anti-ALZAS IgG measured in serum from patients diagnosed with depression, patients diagnosed with clinical AD, and normal controls (the normal serum were obtained from subjects involved in a pre symptomatic breast cancer detection study).
(E) Dot plots of ELISA values for anti.ALZAS endogenous antibody measured in serum obtained from normal control subjects, clinically diagnosed and autopsy confirmed AD positive controls (mixed) and from subjects over 65 years old who have no clinical symptoms of AD, selected at random from available sera (screening).

FIG. 5A-D

(A). Isolation of ALZAS from cortex obtained from a single autopsy confirmed AD patient. The protein was affinity purified on anti ALZab2 antibody linked to CNBR-sepharose. The gel was blotted onto nylon a nylon membrane and the protein detected with a chemiluminescent detection system.
(B) Silver stain of SDS gel: proteins obtained following affinity purification of ALZAS from serum from a late stage AD patient. In addition to ALZAS, ALZAS bound to various immunoglobulins and to APOE was detected on the silver stained gel and confirmed by specific-staining duplicate gels
(C) Detection of ALZAS-IgG complexes present on gel of SB with anti-human IgG (Fc fragment).
(D) Western blot of cationic non SDS polyacrylamide gel following electrophoresis of serum from (a) a patient with sporadic AD, and (b) a patient with swedish mutation AD. The gels were reacted first with anti ALZASab1 and then with the chemiluminescence system. ALZAS was detected in a and ALZAS2 in b. The upper band in a is ALZAS and in b is ALZAS2 both bound to human IgG. ALZAS and ALZAS2 have slightly different isoelectric points which are consistent with how they appear on the gels.

1-5. (canceled)

6. An antibody against SEQ ID NO: 4.

7-10. (canceled)
